SEQUENCE LISTING (14 sequences)

SEQ ID NO: 1 - protein, 387 aa, Artificial Sequence
  synthetic PTX3 modified at positions 18-23 by adding six histidines
MHLPAILLCALWSAVVAHHHHHHETSDDYELMYVNLDNEIDNGLHPTEDPTPCDCRQEHSEWDKLFIMLENSQMREGMLLQATDDVLRGELQRLRAELGRLAGGMARPCAAGGPADARLVRALEPLLQESRDASLRLARLEDAEARRPEATVPGLGAVLEELRRTRADLSAVQSWVARHWLPAGCETAIFFPMRSKKIFGSVHPVRPMKLESFSTCIWVKATDVLNKTILFSYGTKWNPYEIQLYLSSQSLVLVVGGKENKLAADTVVSLGRWSHLCGTWSSEQGSMSLWANGELVATTVEMAKSHSVPEGGLLQIGQEKNGCCVGGGFDESLAFSGRITGFNIWDRVLSEEEIRASGGVESCHIRGNVVGWGVTEIQAHGGAQYVS

SEQ ID NO: 2 - protein, 387 aa, Artificial Sequence
  synthetic PTX3 modified at the C-terminal end by adding six histidines
MHLPAILLCALWSAVVAETSDDYELMYVNLDNEIDNGLHPTEDPTPCDCRQEHSEWDKLFIMLENSQMREGMLLQATDDVLRGELQRLRAELGRLAGGMARPCAAGGPADARLVRALEPLLQESRDASLRLARLEDAEARRPEATVPGLGAVLEELRRTRADLSAVQSWVARHWLPAGCETAIFFPMRSKKIFGSVHPVRPMKLESFSTCIWVKATDVLNKTILFSYGTKWNPYEIQLYLSSQSLVLVVGGKENKLAADTVVSLGRWSHLCGTWSSEQGSMSLWANGELVATTVEMAKSHSVPEGGLLQIGQEKNGCCVGGGFDESLAFSGRITGFNIWDRVLSEEEIRASGGVESCHIRGNVVGWGVTEIQAHGGAQYVSHHHHHH

SEQ ID NO: 3 - protein, 387 aa, Artificial Sequence
  synthetic PTX3 modified at the C-terminal end by adding six histidines
MHLLAILFCALWSAVLAENSDDYDLMYVNLDNEIDNGLHPTEDPTPCDCGQEHSEWDKLFIMLENSQMRERMLLQATDDVLRGELQRLREELGRLAESLARPCAPGAPAEARLTSALDELLQATRDAGRRLARMEGAEAQRPEEAGRALAAVLEELRQTRADLHAVQGWAARSWLPAGCETAILFPMRSKKIFGSVHPVRPMRLESFSACIWVKATDVLNKTILFSYGTKRNPYEIQLYLSYQSIVFVVGGEENKLVAEAMVSLGRWTHLCGTWNSEEGLTSLWVNGELAATTVEMATGHIVPEGGILQIGQEKNGCCVGGGFDETLAFSGRLTGFNIWDSVLSNEEIRETGGAESCHIRGNIVGWGVTEIQPHGGAQYVSHHHHHH

SEQ ID NO: 4 - protein, 387 aa, Artificial Sequence
  synthetic PTX3 modified at positions 18-23 by adding six histidines
MHLLAILFCALWSAVLAHHHHHHENSDDYDLMYVNLDNEIDNGLHPTEDPTPCDCGQEHSEWDKLFIMLENSQMRERMLLQATDDVLRGELQRLREELGRLAESLARPCAPGAPAEARLTSALDELLQATRDAGRRLARMEGAEAQRPEEAGRALAAVLEELRQTRADLHAVQGWAARSWLPAGCETAILFPMRSKKIFGSVHPVRPMRLESFSACIWVKATDVLNKTILFSYGTKRNPYEIQLYLSYQSIVFVVGGEENKLVAEAMVSLGRWTHLCGTWNSEEGLTSLWVNGELAATTVEMATGHIVPEGGILQIGQEKNGCCVGGGFDETLAFSGRLTGFNIWDSVLSNEEIRETGGAESCHIRGNIVGWGVTEIQPHGGAQYVS

SEQ ID NO: 5 - protein, 381 aa, Artificial Sequence
  synthetic PTX3 biotinylated randomly with 1-100 biotins
MHLPAILLCALWSAVVAETSDDYELMYVNLDNEIDNGLHPTEDPTPCDCRQEHSEWDKLFIMLENSQMREGMLLQATDDVLRGELQRLRAELGRLAGGMARPCAAGGPADARLVRALEPLLQESRDASLRLARLEDAEARRPEATVPGLGAVLEELRRTRADLSAVQSWVARHWLPAGCETAIFFPMRSKKIFGSVHPVRPMKLESFSTCIWVKATDVLNKTILFSYGTKWNPYEIQLYLSSQSLVLVVGGKENKLAADTVVSLGRWSHLCGTWSSEQGSMSLWANGELVATTVEMAKSHSVPEGGLLQIGQEKNGCCVGGGFDESLAFSGRITGFNIWDRVLSEEEIRASGGVESCHIRGNVVGWGVTEIQAHGGAQYVS

SEQ ID NO: 6 - protein, 381 aa, Artificial Sequence
  synthetic PTX3 biotinylated randomly with 1-100 biotins
MHLLAILFCALWSAVLAENSDDYDLMYVNLDNEIDNGLHPTEDPTPCDCGQEHSEWDKLFIMLENSQMRERMLLQATDDVLRGELQRLREELGRLAESLARPCAPGAPAEARLTSALDELLQATRDAGRRLARMEGAEAQRPEEAGRALAAVLEELRQTRADLHAVQGWAARSWLPAGCETAILFPMRSKKIFGSVHPVRPMRLESFSACIWVKATDVLNKTILFSYGTKRNPYEIQLYLSYQSIVFVVGGEENKLVAEAMVSLGRWTHLCGTWNSEEGLTSLWVNGELAATTVEMATGHIVPEGGILQIGQEKNGCCVGGGFDETLAFSGRLTGFNIWDSVLSNEEIRETGGAESCHIRGNIVGWGVTEIQPHGGAQYVS

SEQ ID NO: 7 - DNA, 1244 bp, Artificial Sequence
  synthetic PTX3 modified to encode six histidines
ctaggattcggatcactgtagagtctcgcttcttcccctgcggctgcgaacgaaatttcgcctctccagcaatgcacctccctgcgatcctgctttgtgctctctggtctgcagtagtggctcatcaccatcaccatcatgagacctcggatgactacgagctcatgtatgtgaatttggacaacgaaatagacaatggacttcatcccaccgaggaccccacgccatgcgactgccgccaggagcactcggagtgggacaagctgttcatcatgctggagaactcgcagatgcgggagggcatgctgttgcaggccaccgacgacgtcctccgtggagagctgcagcggctgcgggcagagctggggcggctggcgggcggcatggcgaggccgtgcgcagccggtggccccgcagacgccaggctggtgcgggcgctggagccgctgctgcaggagagccgtgacgcgagcctcaggctggcgcgcctggaggacgcggaggcgcggcgacccgaggcgacagtgcctggcctaggcgctgtgctggaggaactgcggcggacgcgcgccgacctgagcgccgtgcagagctgggtcgcccgccactggctgcccgcaggttgtgaaacagcaattttcttcccaatgcgttcgaagaagatttttggaagcgtgcatcctgtgagaccaatgaagcttgaatcttttagtacttgcatttgggtcaaagccacagatgtattaaacaaaaccatcctgttttcttatggcacaaagtggaacccctatgagattcagctgtacctcagttcccagtccctagtgttggtggtgggtggaaaggagaacaagctggctgcagacactgtggtgtccctggggaggtggtcccacctgtgtggcacctggagttcagagcaggggagcatgtccctgtgggcaaacggggagctggtggctaccactgtagagatggccaaaagtcactctgttcctgagggtggactcctacagattggccaagaaaagaatggttgctgtgtaggtgggggctttgacgaatcattagcattttctggaagaatcacaggcttcaatatctgggatcgggttctcagcgaggaggagatacgggccagtggaggagtcgaatcctgtcacatccggggaaatgtcgtcgggtggggagtcacagagattcaggcgcacggaggagcccagtatgtttcttaatctagagag

SEQ ID NO: 8 - DNA, 1189 bp, Artificial Sequence
  synthetic PTX3 modified to encode six histidines
ctagaattcctaccatgcacctccctgcgatcctgctttgtgctctctggtctgcagtagtggctgagacctcggatgactacgagctcatgtatgtgaatttggacaacgaaatagacaatggacttcatcccaccgaggaccccacgccatgcgactgccgccaggagcactcggagtgggacaagctgttcatcatgctggagaactcgcagatgcgggagggcatgctgttgcaggccaccgacgacgtcctccgtggagagctgcagcggctgcgggcagagctggggcggctggcgggcggcatggcgaggccgtgcgcagccggtggccccgcagacgccaggctggtgcgggcgctggagccgctgctgcaggagagccgtgacgcgagcctcaggctggcgcgcctggaggacgcggaggcgcggcgacccgaggcgacagtgcctggcctaggcgctgtgctggaggaactgcggcggacgcgcgccgacctgagcgccgtgcagagctgggtcgcccgccactggctgcccgcaggttgtgaaacagcaattttcttcccaatgcgttcgaagaagatttttggaagcgtgcatcctgtgagaccaatgaagcttgaatcttttagtacttgcatttgggtcaaagccacagatgtattaaacaaaaccatcctgttttcttatggcacaaagtggaacccctatgagattcagctgtacctcagttcccagtccctagtgttggtggtgggtggaaaggagaacaagctggctgcagacactgtggtgtccctggggaggtggtcccacctgtgtggcacctggagttcagagcaggggagcatgtccctgtgggcaaacggggagctggtggctaccactgtagagatggccaaaagtcactctgttcctgagggtggactcctacagattggccaagaaaagaatggttgctgtgtaggtgggggctttgacgaatcattagcattttctggaagaatcacaggcttcaatatctgggatcgggttctcagcgaggaggagatacgggccagtggaggagtcgaatcctgtcacatccggggaaatgtcgtcgggtggggagtcacagagattcaggcgcacggaggagcccagtatgtttctcatcatcatcatcatcattaagcggccgcgag

SEQ ID NO: 9 - DNA, 29 bp, Artificial Sequence
  synthetic primer
ctagaattcggatcactgtagagtctcgc

SEQ ID NO: 10 - DNA, 54 bp, Artificial Sequence
  synthetic primer
ctcgtagtcatccgaggtctcatgatggtgatggtgatgagccactactgcaga

SEQ ID NO: 11 - DNA, 21 bp, Artificial Sequence
  synthetic primer
gagacctcggatgactacgag

SEQ ID NO: 12 - DNA, 30 bp, Artificial Sequence
  synthetic primer
ctctctagattaagaaacatactgggctcc

SEQ ID NO: 13 - DNA, 29 bp, Artificial Sequence
  synthetic primer
ctagaattcctaccatgcacctccctgcg

SEQ ID NO: 14 - DNA, 47 bp, Artificial Sequence
  synthetic primer
ctcgcggccgcttaatgatgatgatgatgatgagaaacatactgggc

(19) United States
(12) Patent Application Publication
(10) Pub. No.: US 2009/0110666 A1
(43) Pub. Date: Apr. 30, 2009

(54) Long pentraxin ptx3 functional derivatives for preparing an autologous vaccine for the treatment of tumours

(76) Inventors: Rita De Santis, Roma (IT); Salvatori Giovanni, Roma (IT)

Correspondence Address: NIXON & VANDERHYE, PC, 901 NORTH GLEBE ROAD, 11TH FLOOR, ARLINGTON, VA, 22203, US

(21) Appl. No.: 10/506,046

(22) PCT Filed: Feb. 25, 2003
(86) PCT No.: PCT/IT2003/000104

§ 371 Date: Aug. 30, 2004

(30) Foreign Application Priority Data
Feb. 28, 2002 (IT) RM2002A000109

(51) Int. Cl. A61K 35/12; C07K 14/47; A61K 38/17; A61P 35/00; C12N 13/00; C07H 21/04

(52) U.S. Cl. 424/93.7; 530/350; 536/23.1; 514/12; 435/173.1

(57) ABSTRACT

The invention described herein refers to derivatives of the long pentraxin PTX3 with the sequence indicated in the text, capable of binding to the membranes of inactivated tumour cells. The inactivated tumour cells, bearing on their surface a derivative of PTX3 are used to prepare an autologous vaccine for the treatment of tumours.

The invention described herein relates to analogues of the long pentraxin PTX3 (PTX3) and their use for the preparation of a vaccine for the treatment of tumours.

The spontaneous activation of a response of the immune system against a tumour is often ineffective. The tumour, in fact, is capable of concealing itself from host's immune system through reduced expression of its own antigens or through the ineffective presentation of said antigens. It is known that both the class I major histocompatibility complex (MHC I) and molecules with co-stimulatory activity such as CD80 and CD86 are poorly or not all expressed by tumour cells. The tumour, moreover, is capable of secreting cytokines with an immunosuppressive activity such as IL-10 and TGFβ, the function of which is to de-energise lymphocytes activated against associated tumour antigens. On the whole, the tumour induces a state of immunological tolerance in the host. The aim of vaccine therapy for cancer is to disrupt this state of tolerance and activate an immune response against the tumour.

The methods of cancer vaccine therapy involve the use of tumour cells modified, for example, by cytokines, by co-stimulatory molecules, bacteria or toxins, for the purposes of modifying the tumour cells and making them recognisable or capable of being processed by the immune system.

PTX3 is a protein which is expressed in various cell types (Bottazzi et al., J. Biol Chem; 272: 32817-32823, 1997) particularly in mononuclear phagocytes and endothelial cells, after exposure to the inflammatory cytokines, Interleukin 1 beta (IL-1 beta) and Tumour Necrosis Factor alpha (TNF-alpha).

This protein consists of two structural domains, an N-terminal unrelated to any known molecule, and a C-terminal similar to the short pentraxins such as C-reactive protein (CRP).

The PTX3 gene is located on mouse chromosome 3, in a region similar to the human region 3q (q24-28), in agreement with the documented location of hPTX3 in the region 3q 25.

In addition, mouse PTX3 (mPTX3) (Introna M. et al., Blood 87 (1996, 1862-1872) is very similar to hPTX3 on the basis of its organisation, location and sequence (Breviario F. et al., J. Biol. Chem. 267:22190, 1992).

In particular, the degree of identity between the sequences is 82% between the human gene and the mouse gene, and as much as 92% if the conservative substitutions are considered.

The high degree of similarity between the sequence of hPTX3 and that of mPTX3 is a sign of the high degree of conservation of the pentraxins during evolution (Pepys M. B., Baltz M. L., Adv. Immunol: 34:141, 1983).

For a review of the pentraxins, see H. Gewurz et al., Current Opinion in Immunology, 1995, 7:54-64.

Previous uses of PTX3 are already known.

In WO99/32516, filed in the name of the applicant, the use of long pentraxin PTX3 is described for the therapy of diseases of an infectious, inflammatory or tumoral type. In WO99/32516 a gene therapy method is described in which the anticancer activity of PTX3 is described.

U.S. Pat. No. 5,767,252 describes a growth factor of neuronal cells belonging to the pentraxin family (see also the literature cited therein). This patent refers to the neurobiology sector.

To date the use of PTX3, or its analogues, for the preparation of a vaccine for the treatment of tumours has never been described.

It is well known in the medical field that there is a need for the availability of new vaccines for the treatment of tumours.

It has now been found that the derivatives of the long pentraxin PTX3 lend themselves to use for preparing a vaccine for the treatment of tumours.

The object of the invention described herein is therefore a derivative of murine PTX3 with amino-acid sequence Seq. Id. No. 1.

A further object of the invention described herein is a derivative of murine PTX3 with amino-acid sequence Seq. Id. No. 2.

A further object of the invention described herein is a derivative of human PTX3 with amino-acid sequence Seq. Id. No. 3.

A further object of the invention described herein is a derivative of human PTX3 with amino-acid sequence Seq. Id. No. 4.

A further object of the invention described herein is a derivative of murine PTX3 biotinylated at random, with 1-100 molecules of biotin per single protein of PTX3, with amino-acid sequence Seq. Id. No. 5.

A further object of the invention described herein is a derivative of human PTX3 biotinylated at random, with 1-100 molecules of biotin per single protein of PTX3, with amino-acid sequence Seq. Id. No. 6.

A further object of the invention described herein is a Murine PTX3 cDNA having sequence Seq. Id. No. 7.

A further object of the invention described herein is a Murine PTX3 cDNA having sequence Seq. Id. No. 8.

A further object of the invention described herein is an autologous vaccine containing inactivated tumour cells of a solid or haematological tumour, bearing on their surface a derivative of PTX3 with amino-acid sequence Seq. Id. No. 1-6, and possibly an adjuvant.

A further object of the invention described herein is a procedure for preparing an autologous vaccine, consisting of the following stages:
taking tumour cells, by means of known methods, from a patient suffering from a solid or haematological tumours;
inactivation, in vitro, of the tumour cells by means of known methods, e.g. radiation, in order to inhibit their proliferative ability;
treatment of the inactivated tumour cells with liposomes of the lipid chelating agent NTA-DOGS, as described in the experimental part here below;
further treatment of the tumour cells with a derivative of PTX3 with amino-acid sequence Seq. Id. No. 1, 2, 3 or 4, in order to bind said derivative of PTX3 to the membranes of said tumour cells, which are used for the therapeutic vaccination.

A further object of the invention described herein is a process for preparing an autologous vaccine consisting of the following stages:
taking tumour cells from a patient suffering from a solid or haematological tumour;
inactivation, in vitro, of the tumour cells by means of known methods, e.g. radiation, in order to inhibit their proliferative ability;
biotinylation of the inactivated tumour cells and incubation of said cells with avidin, as described in the experimental part here below;
binding of a derivative of biotinylated PTX3, with amino-acid sequence Seq. Id. No. 5 or 6, to the membranes of the tumour cells in the previous stage, which are used for the therapeutic vaccination.

A further object of the invention described herein is the use of a vaccine prepared with the procedures outlined above for the preparation of a medicine which can be administered, for instance, by the subcutaneous, intravenous or intra-lymph-nodal routes for the treatment of tumours.

A further object of the invention described herein is the use of a derivative of PTX3 with amino-acid sequence Seq. Id. No. 1-6, bound to the surface of the inactivated tumour cells of a solid or haematological tumour, for the preparation of an autologous vaccine which can be administered by the subcutaneous, intravenous or intra-lymph-nodal or other routes for the treatment of tumours.

A further object of the invention described herein is the use of a vaccine, prepared with a derivative of PTX3 with amino-acid sequence Seq. Id. No. 1-6, in which said derivative is bound to the surface of the inactivated tumour cells of a solid tumour, for the preparation of a medicine which can be administered by the subcutaneous, intravenous, intra-lymph-nodal or other routes for the treatment of tumours.

The tumour vaccine according to the invention described herein may contain one or more adjuvants that induce a non-specific immune response.

Examples of adjuvants are Freund's complete adjuvant, Freund's incomplete adjuvant, bacterial preparations such as, for example, BCG, preparations of bacterial components such as tuberculin, naturally-occurring macromolecular substances such as mannan yeast, alum, synthetic adjuvants such as “Titer Max Gold” and the like.

Other adjuvants can obviously also be used.

The vaccine according to the invention can be inoculated in either the presence or absence of the adjuvant.

The following examples further illustrate the invention.

Engineering of PTX3 cDNA for the Production of Recombinant Protein Containing a 6 Histidine Domain.

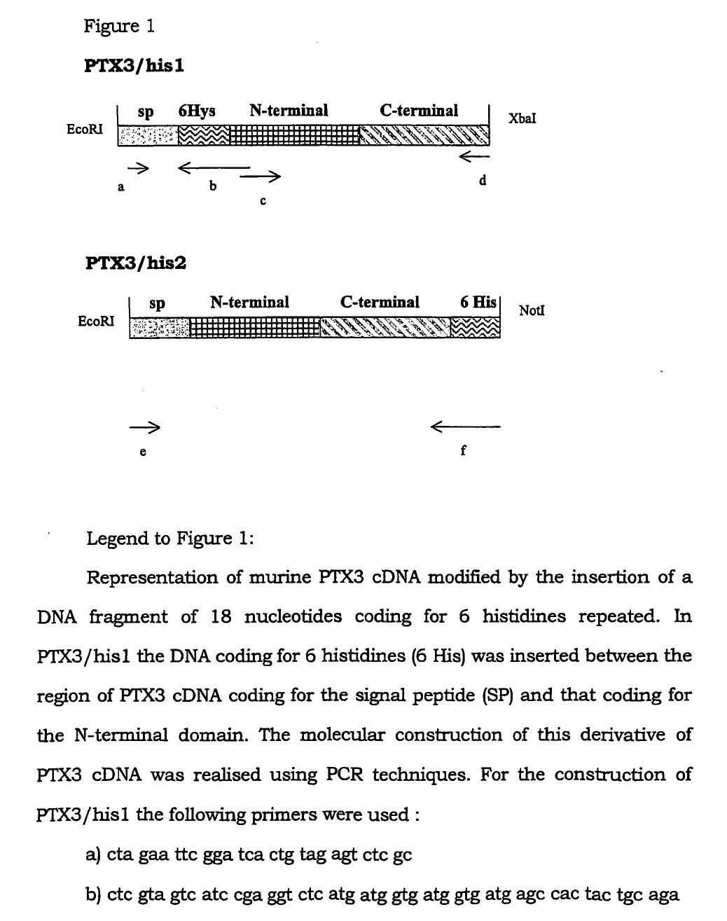

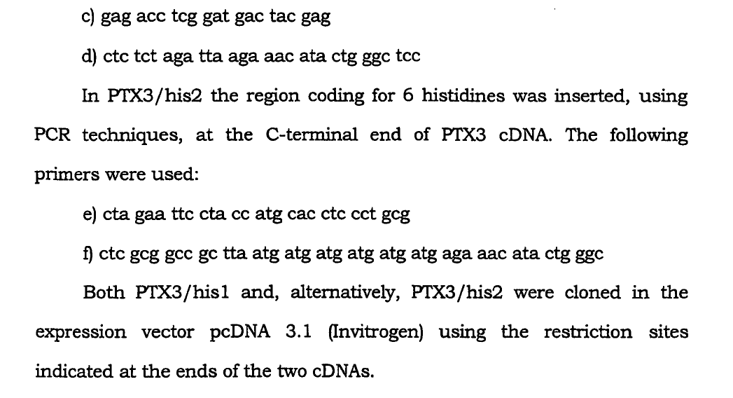

Murine PTX3 cDNA (Introna M. et al. Blood 87 (1996) 1862-1872) was modified by the introduction of a sequence of 18 nucleotides coding for 6 histidines between the signal peptide and the N-terminal domain of PTX3. The insertion of the 18 nucleotides in the open reading frame (ORF) of PTX3 was obtained using the recombinant PCR techniques described in Recombinant PCR (Russel Higuchi, PCR Protocols, edited by M. Innis, D. H. Gelfand, J. J. Sninsky, T. J. White, 1990, San Diego USA) (FIG. 1).

Murine PTX3 cDNA thus modified (Seq. Id. No. 7) was cloned in the plasmid expression vector pcDNA 3.1 (Invitrogen) using the EcoRI and XbaI restriction sites (Ausubel F. M. et al., 1987, Current Protocols in Molecular Biology, Wiley Interscience, New York). This plasmid vector was called pPTX3/his1.

Similar PCR techniques to those mentioned above were used to introduce the 18 nucleotides coding for 6 histidine at the C-Terminal end of murine PTX3 cDNA (FIG. 1). The murine PTX3 cDNA thus modified (Seq. Id. No. 8) was cloned in the plasmid expression vector pcDNA 3.1 (Invitrogen) using the restriction sites EcoRI e NotI. The plasmid vector was called pPTX3/his2.

Production and Purification of Derivatives PTX3/his1 and PTX3/his2

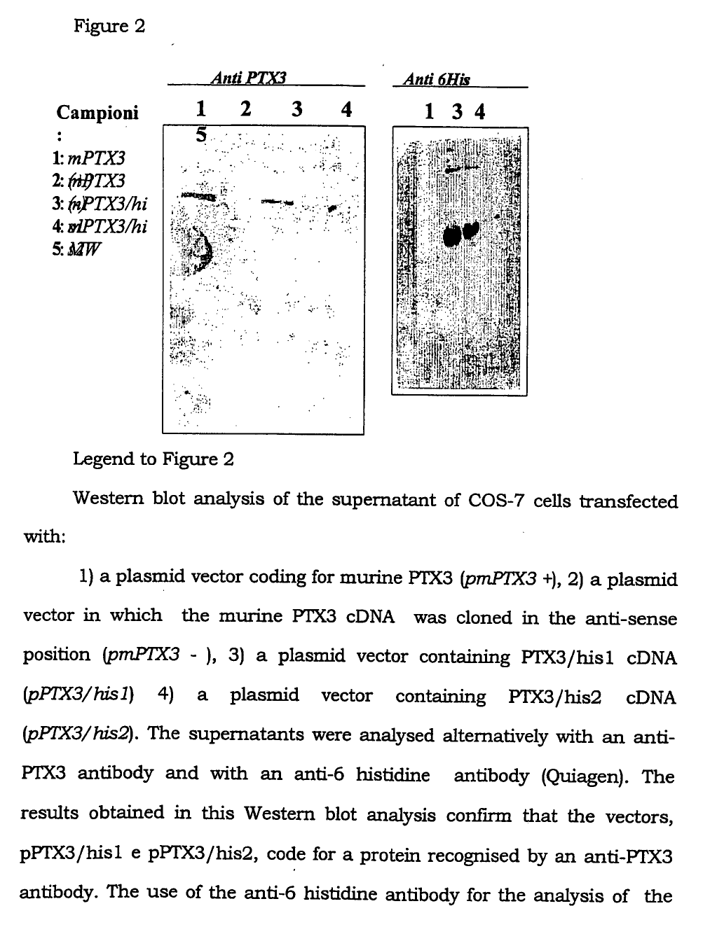

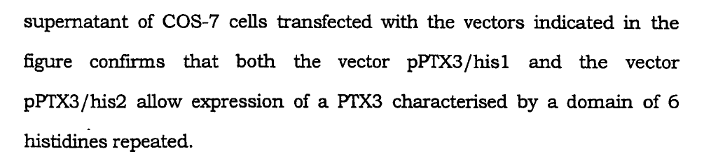

The plasmid vectors pPTX3/his1 and pPTX3/his2 were used for the transfection of COS7 cells with lipofectamine 2000 (Invitrogen) (Ciccarone et al., 1999 FOCUS 21, 54). After transfection with one of the two plasmids, these cells release an amino-acid sequence of the murine recombinant PTX3 into the culture medium (DMEM GIBCO) (the plasmid vector pPTX3/his1 codes for Seq. Id. No. 1, while plasmid vector pPTX3/his2 codes for Seq. Id. No. 2) recognised both by anti-PTX3 antibodies and by anti-histidine antibodies (Quiagen) (FIG. 2). In the transfections of COS7 cells with the plasmid pPTX3/his1, the protein produced (Seq. Id. No. 1) was called PTX3his1. Likewise, in the transfections of COS7 cells with plasmid pPTX3/his2, the protein produced (Seq. Id. No. 2) was called PTX3his2.

PTX3his1 and PTX3his2 were purified by affinity chromatography, using Amersham Pharmacia Biotech columns (Histrap Kit). The passage of the dialysed supernatant of COS-7 cells transfected with one of the two vectors and the subsequent elution of the protein with a discontinuous gradient of imidazole from these columns, permits the recovery of approximately 60-80% of the recombinant PTX3 produced.

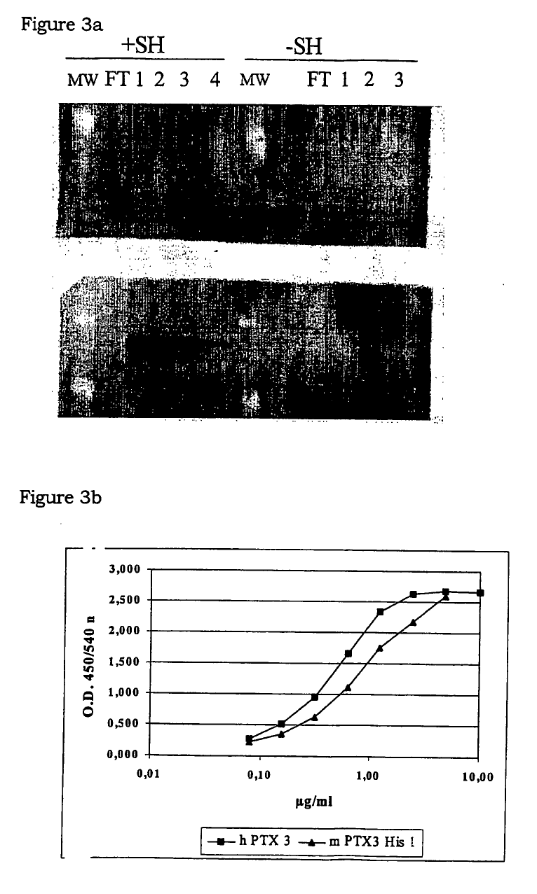

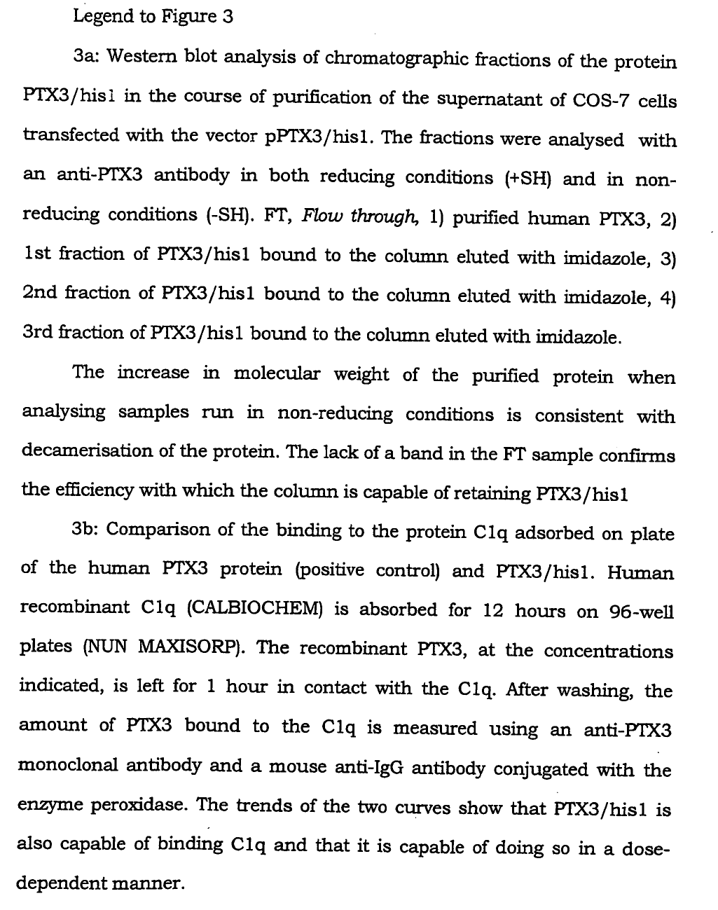

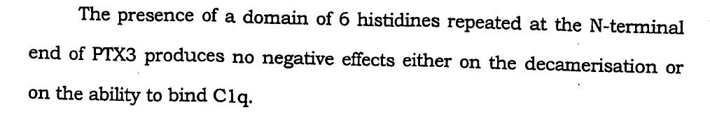

The protein PTX3his1 shows an ability to decamerise (FIG. 3a) and bind C1q (FIG. 3b) in a similar way to that described for the naturally occurring protein of PTX3.

Likewise it is possible to prepare human recombinant PTX3 (sequences Seq. Id. Nos. 3 and 4), starting from cDNA of human PTX3 (Breviario F. et al., J. Biol. Chem. 267:22190, 1992).

Production and Purification of Naturally Occurring Murine PTX3 to be Used for Biotinylation.

Murine PTX3 cDNA (Introna M. et al., Blood 87 (1996) 1862-1872) was subcloned in the expression vector pcDNA 3.1 (Invitrogen) e subsequently transfected in COS7 cells using lipofectamine 2000 (Invitrogen) (Ciccarone et al., 1999 FOCUS 21, 54).

The recombinant protein thus obtained was purified from the culture supernatant of the COS7 cells by means of affinity chromatography, using an anti-PTX3 monoclonal antibody conjugated to protein G, with the procedure described by Bottazzi et al., J. Biol. Chem. 272(52):32817-32823, 1997.

Likewise, it is possible to prepare the human recombinant PTX3 protein starting from the expression of human cDNA in COS7 cells (Breviario F. et al., J. Biol. Chem. 267:22190, 1992).

Biotinylation of Naturally Occurring PTX3 Protein and the Membrane Proteins of Tumour Cells

Biotin is a 244-dalton molecule capable of binding avidin and streptoavidin molecules with high affinity. Biotin was bound to amino-acid residues of human and mouse PTX3, or proteins of cell membranes of inactivated tumour cells using the chemical derivative NHS-LC-Biotin (PIERCE) (Altin et al., Anal Biochem 224: 382-389, 1995). The binding of biotin molecules both to the membranes of tumour cells and to recombinant PTX3 protein makes it possible to anchor PTX3 to the tumour cell. The molecules of avidin added to the mixture of PTX3 and tumour cells act as a molecular bridge between the biotins present on the cell membrane and those bound to the PTX3 amino acids.

Modification of the P815 Tumour Cell Membrane with Liposomes of the Lipid Chelating Agent NTA-DOGS.

The lipid chelating agent NTA-DOGS (Avanti Polar Lipids Inc.) was prepared as a liposomal suspension with liposomes with a mean diameter of approximately 500 nm. As a result of the fusion of the liposomes with the cell membranes of murine P815 mastocytoma cells, NTA-DOGS is intercalated in the lipid bilayer via its hydrophobic portion and exposes, on the cell surface, the polar head of nitrolotriacetic acid capable of binding any peptide or protein containing 6 histidine domains (Broekhoven et al. 2000 J. Immunology 164: 2433-2443). The efficiency of incorporation of the lipid chelating agent in the bilayer of the membrane of the P815 tumour cell line was measured using a 6-histidine peptide conjugated to a biotin molecule. FACS (fluorescence activated cell sorter) analysis of the P815 cells treated with liposomes of NTA-DOGS (P815-NTA), with the biotinylated peptide and lastly with fluorescinated streptoavidin, revealed an approximately 100-fold increase in the fluorescent signal compared to controls (P815 treated with the biotinylated peptide alone).

The Protein PTX3/his1 is Capable of Binding to the Membrane Surface of Tumour Cells Treated with Liposomes of the Lipid Chelating Agent NTA-DOGS.

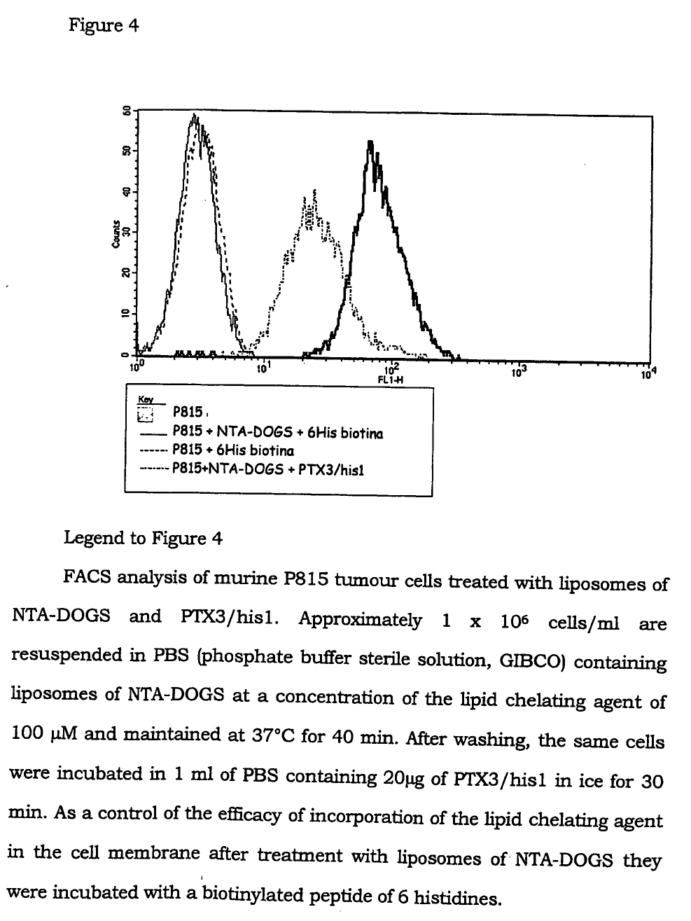

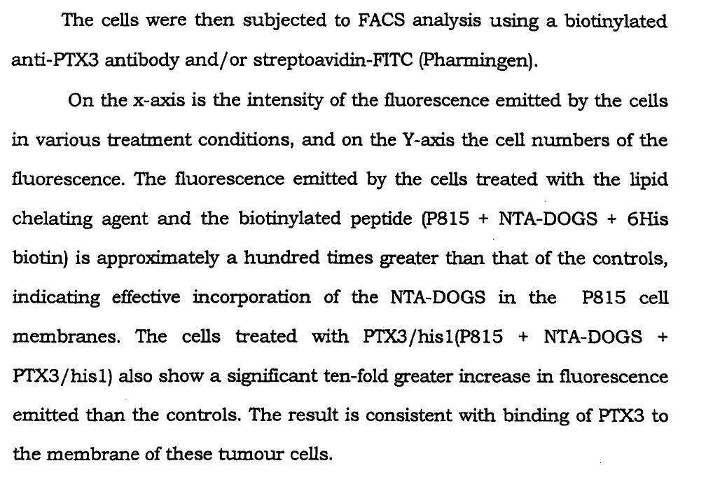

The protein PTX3/his 1 purified from the supernatant of COS-7 cells and incubated with P815-NTA cells is capable of binding to their membrane surface. FACS analysis of P815-NTA cells using anti-PTX3 antibodies revealed a 10-fold greater fluorescent signal than P815 controls not treated with the recombinant protein (FIG. 4). This result confirms that binding of PTX3/his1 to the P815 cell membrane has taken place.

### EXAMPLE 1

Preparation of an Autologous Anticancer Vaccine by Means of the Use of a Derivative of PTX3 with Amino-Acid Sequence Seq. Id. No. 1, 2, 3 or 4, Bound to Tumour Cells

A) Tumour cells (10-100 million) are taken, by means of known methods, from a patient suffering from a solid tumour.

B) These tumour cells are inactivated with known methods, in vitro, in order to inhibit their proliferative ability, for example by radiation.

C) The inactivated tumour cells are treated with liposomes of the lipid chelating agent NTA-DOGS (50-250 μM).

D) The tumour cells are further treated with a derivative of PTX3 (50-500 μg/ml) with amino-acid sequence Seq. Id. No. 1, 2, 3 or 4, in order to bind said derivative of PTX3 to the membranes of said tumour cells.

E) An aliquot of tumour cells thus modified is subjected to FACS analysis to verify the presence of the PTX3 derivative on their membranes.

F) The modified tumour cells, with the PTX3 derivative bound to the membranes, are inoculated into the patient from whom they have come (autologous vaccine) by means of administration via the subcutaneous, intravenous, intra-lymph-nodal or other routes.

### EXAMPLE 2

### Preparation of an Autologous Anticancer Vaccine by Means of the Use of a PTX3 Derivative with Amino-Acid Sequence Seq. Id. No. 5 or 6, Bound to Tumour Cells

a) The tumour cells (10-100 million) are taken, by means of known methods, from a patient suffering from a tumour.

b) These tumour cells are inactivated, by means of known methods, in vitro, in order to inhibit their proliferative activity, for example, by radiation.

c) The inactivated tumour cells are subjected to biotinylation (100-1000 biotins/cell).

d) The biotinylated tumour cells are incubated with avidin (10-100 μg/ml).

e) To the cell membrane of the tumour cells incubated with avidin (as in para. “d”) is bound a biotinylated PTX3 derivative (50-500 μg/ml) with amino-acid sequence Seq. Id. No. 5 or 6.

f) The modified tumour cells with the PTX3 bound to the membranes (as in para. “e”) are inoculated into the patient from whom they have come (autologous vaccine) by means of administration via the subcutaneous, intravenous, intra-lymph-nodal or other routes.

### EXAMPLE 3

The subcutaneous inoculation, in syngenic mice, of P815 cells modified ex-vivo with PTX3 on the cell membranes induces a significant reduction in the tumour growth rate.

As a model for the in-vivo study, the murine mastocytoma P815 line was used, to which the modified PTX3 was bound. The aim of the experiment was to assess the frequency of rejection or any reduction in the growth rate of the modified tumour compared to controls not treated with PTX3/his1.

Syngenic DBA2J mice were inoculated subcutaneously with 1×10⁵ P815 cells bearing the protein PTX3/his1 on the cell membranes.

The results obtained, reported in Table 1, show that the tumour cells modified by the presence of the PTX3/his1 protein on their membranes, in DBA2J mice, grow more slowly than untreated parental cells or parental cells treated only with the lipid chelating agent NTA-DOGS.

Preliminary data obtained in further experiments show that the vaccine according to the present invention stimulates an immunogenic response against the tumour which the modified cells come from.

TABLE 1

| | 13 days | 15 days | 17 days | 20 days | 22 days | N. of |
|---|---|---|---|---|---|---|
| Animal | mean ± | mean ± | mean ± | mean ± | mean ± | animals/ |
| groups | sd (mm³) | sd (mm³) | sd (mm³) | sd (mm³) | sd (mm³) | group |
| P815 | 330.2 +/− 182.5 | 599.5 +/− 278.7 | 956.8 +/− 391.4 | 1422.8 +/− 530.8 | 2325.1 +/− 1056.0 | 10 |
| P815 + NTA- | 355.9 +/− 222.8 | 420.4 +/− 362.4 | 626.8 +/− 434.2 | 851.7 +/− 590.5 | 967.1 +/− 519.4 | 9 |
| DOGS + PTX | | | | | | |
| 3his1 | | | | | | |
| P815 + NTA- | 379.1 +/− 207.2 | 628.9 +/− 291.6 | 1198.3 +/− 436.4 | 1644.7 +/− 893.6 | 2122.9 +/− 581.8 | 7 |
| DOGS | | | | | | |

Legend to Table 1
DBA2J mice were inoculated subcutaneously with 1 × 10⁵ murine P815 tumour cells. One group of animals (n = 7) was inoculated with P815 cells modified by treatment with liposomes of NTA-DOGS (P815 + NTA-DOGS). A second group of animals (n = 9) was inoculated with P815 cells treated with the lipid chelating agent and with PTX3/his1 (20 μg/ml) (P815 + NTA-DOGS + PTX3/his1). A third group of animals (n = 10) was treated with parental P815 cells (P815). Tumour sizes were measured in the three weeks following inoculation of the cells on the days indicated in the table, by direct measurement with a Vernier calliper. The calculation of tumour size in mm³ was done using the formula [(width² × length)/2].

1. Derivative of murine PTX3 with amino-acid sequence Seq. Id. No. 1.

2. Derivative of murine PTX3 with amino-acid sequence Seq. Id. No. 2.

3. Derivative of human PTX3 with amino-acid sequence Seq. Id. No. 3.

4. Derivative of human PTX3 with amino-acid sequence Seq. Id. No. 4.

5. Biotinylated derivative of murine PTX3 with amino-acid sequence Seq. Id. No. 5.

6. Biotinylated derivative of human PTX3 with amino-acid sequence Seq. Id. No. 6.

7. Murine PTX3 CDNA having sequence Seq. Id. No. 7.

8. Murine PTX3 CDNA having sequence Seq. Id. No. 8.

9. Autologous vaccine containing inactivated tumour cells of a solid or haematological tumour, bearing on their surface a derivative of PTX3 according to claim 1.

10. Vaccine according to claim 9, which additionally contains an adjuvant.

11. Procedure for the preparation of an autologous vaccine, consisting of the stages of:
taking samples of tumour cells (10-100 million) from a patient suffering from a solid or haematological tumour;
inactivation, in vitro, of the tumour cells, for example, by radiation, in order to inhibit their proliferative ability;
treatment of the inactivated tumour cells with liposomes of the lipid chelating agent NTA-DOGS;
further treatment of the tumour cells with a derivative of PTX3 (50-500 J, G/ML) with amino-acid sequence Seq. Id. No. 1, 2, 3 or 4, in order to bind said derivative of PTX3 to the membranes of said tumour cells.

12. Procedure for preparing an autologous vaccine, consisting of the stages of:
taking samples of tumour cells (10-100 million) from A patient suffering from a solid or haematological tumour;
inactivation, in vitro, of the tumour cells by means of known methods, for example, radiation, in order to inhibit their ability to proliferate
biotinylation of the inactivated tumour cells with 100-1000 biotins/cell, and incubation thereof with avidin;
binding of a derivative of biotinylated PTX3 (50-500, UG/ML) with amino-acid sequence Seq. Id. No. 5 or 6, to the membranes of tumour cells from the previous stage.

13. Use of a derivative according to claim 1, bound to the surface of inactivated tumour cells of a solid or haematological tumour, for the preparation of an autologous vaccine which can be administered by the subcutaneous, intravenous, intra-lymph-nodal or other routes, for the treatment of tumours.

14. Use of the vaccine according to claim 9, for the preparation of a medicine which can be administered by the subcutaneous, intravenous, intra-lymph-nodal or other routes, for the treatment of tumours.

15. Use of the vaccine obtained with the procedure according to claim 11, for the preparation of a medicine which can be administered by the subcutaneous, intravenous or intra-lymph-nodal routes, for the treatment of tumours.
